(19) United States
(12) Patent Application Publication
(10) Pub. No.: US 2005/0124946 A1
(43) Pub. Date: Jun. 9, 2005

(54) Wound irrigation apparatus and method

(76) Inventors: Sergio Landau, Laguna Niguel, CA (US); Marc Taub, Mission Viejo, CA (US); John Staylor, Wilsonville, OR (US)

Correspondence Address: Law Office of Terry L. Miller, 24832 Via San Fernando, Mission Viejo, CA, 92692, US

(21) Appl. No.: 10/729,297

(22) Filed: Dec. 5, 2003

(51) Int. Cl. A61M 1/00

(52) U.S. Cl. 604/317

(57) ABSTRACT

A dispenser cap for combination with a standard flexible irrigation solution bottle to make a portable wound irrigation dispenser includes a collar threadably engageable with the irrigation bottle, and closed at one end by a wall defining a tubular neck. The tubular neck forms a flow path from the bottle to a splash shield including a nozzle for directing irrigation solution. A valve is disposed in the flow path for opening and closing this flow path. A combination apparatus includes the flexible irrigation solution bottle, which may be inverted and squeezed in order to direct a jet of cleansing liquid into a wound. A single use version of the apparatus includes a valve which is closed in a storage and shipping configuration of the apparatus, and an indicator which is freed within the transparent or translucent flexible bottle when the valve is opened, thus irreversibly indicating that the apparatus has been used. An alternative embodiment of the apparatus includes a safety valve which also prevents misuse of the apparatus without the splash shield installed.

### FIELD OF THE INVENTION

This invention relates to wound irrigation and cleansing. More particularly, the present invention relates to a portable wound irrigation liquid dispenser apparatus for delivering a pressurized stream of wound cleansing liquid, and to a method of making and operating such an apparatus.

### RELATED TECHNOLOGY

Wounds, lacerations, abrasions, and other traumatic injuries to the skin are among the most common problems treated in emergency departments. To prevent infection wounds must be cleaned of bacteria, dirt, and other foreign material before repair (i.e., suturing) is attempted. Unfortunately, traditional methods for cleaning wounds frequently result in one or more of: further trauma to injured tissue, inadequate cleansing, safety hazards for the patient, and safety hazards for the healthcare provider. An ideal wound cleansing system would be characterized by:
Efficacy in cleaning wounds of bacteria and foreign material,
Ease and efficiency of use,
Patient and healthcare provider safety, and
Low cost

A common and long-employed method of wound cleaning involves scrubbing a wound with an antiseptic solution, using gauze or a brush to scrub dirt, debris, and bacterial contamination out of the open wound.

However, most antiseptics are toxic to open tissue, and brushes and gauze cause further tissue injury. Deficiencies of this method include impaired healing, increased incidence of infection, and unnecessary scarring.

In recent years, wound irrigation has emerged as the standard of care for wound cleaning. This method is recognized and recommended by most experts and emergency medical textbooks. Wound irrigation involves directing a stream of liquid into the open wound. Sterile saline solution, several hundred milliliters in volume, at pressures of 8-15 psig, is most commonly used. The fluid stream dislodges foreign material from wounds with minimal tissue trauma.

Several irrigation systems and devices are known. One of the most common methods involves attaching an I.V. catheter tip to the end of a 20-60 ml syringe. The healthcare provider pours irrigation fluid into a basin, then repeatedly draws up, directs, and sprays the fluid from the syringe, through the catheter tip, and into the wound. Principle deficiencies of this method include inefficiency of the repeated drawing and spraying action, and potential for backsplash of fluid onto healthcare providers.

Another common, but deficient, irrigation method involves simply puncturing the cap or lid of a plastic bottle of irrigation solution (i.e., saline solution) with a large bore needle, then spraying liquid directly from the punctured bottle. Although this method is quick and easy to perform, deficiencies of this method include a significant potential for injury when puncturing bottles with the needle, and backsplash of contaminated liquid from the wound onto the healthcare worker. Additionally, this method seems to encourage the use of leftover fluid on other patients. This is the case because most saline bottles contain 1000 ml, and wounds generally require less than 500 ml. for adequate cleansing. However, these common saline bottles are not intended for multiple use, a practice which carries risks of cross contamination with viral and bacterial organisms.

A number of devices have been developed to address the problem of backsplash of contaminated liquid onto the healthcare worker. These devices commonly feature a small conical shield around a central nozzle. The devices attach to a luer tip syringe. Although liquid backsplash from a wound is effectively reduced or even eliminated, these devices still suffer from an undesirable inefficiency. That is, these devices require repeated removal of the splash shield, drawing up of the irrigation fluid, replacing the shield, then spraying the irrigation fluid into the wound.

Newer adaptations of some of these conventional devices utilize tubing to connect the syringe setup to either a bag or basin containing the fluid, in order to permit easier refilling of the syringe, without removal of the splash shield. That is, a check valve arrangement in the tubing allows the syringe to by filled, and then allows the irrigation fluid to be discharged into the wound without removal or replacement of a splash shield. Although efficiency of wound treatment is enhanced (albeit at the expense of additional parts and procedural complexity) the need for the repeated actions of aspiration and expulsion of fluid into and from a syringe still remains.

Another conventional wound irrigation device addresses the disadvantage of repeatedly having to fill and discharge a syringe by use of an adapter that allows a splash shield to be “spiked” directly into an IV bag. With this device, healthcare providers need only squeeze the IV bag to expel the solution. However, deficiencies still remain. With this apparatus, spillage of the irrigation liquid may occur whenever the bag is set down during a procedure, or afterwards when the bag and leftover fluid are discarded into a waste container. Additionally, this device requires “spiking” a sharp tip into an IV bag, creating an injury hazard for the healthcare provider.

Yet another version of irrigation involves an aerosolized or pressurized canister of irrigation fluid. Deficiencies of this apparatus and method include a lack of backsplash protection, an inability to monitor amount of fluid expelled, and a potential for reuse of the apparatus on multiple patients.

### SUMMARY OF THE INVENTION

In view of the deficiencies of the conventional technology, an object for this invention is to avoid or reduce at least one of these deficiencies.

It is an object of this invention to provide a portable wound cleansing device that includes a nozzle and splash shield which directs a pressurized jet or stream of wound cleaning liquid upon and into a wound with good control and accuracy of the delivered stream of cleaning liquid.

It is yet another object of this invention to provide a wound cleansing apparatus that effectively removes foreign materials, including for example particles and bacteria, from a wound.

It is yet another object of this invention to provide a wound cleansing apparatus that attaches directly to standard plastic bottles of irrigation fluid (i.e., saline solution), thus eliminating the need to repeatedly aspirate and eject fluid into and from a syringe.

It is yet another object of this invention to provide a wound cleansing device that protects the healthcare provider from fluid splashing off the wound during irrigation.

It is yet another object of this invention to provide a wound cleansing device that does not required the use of syringes, needles, spike adapters, or other hazardous objects.

It is yet another object of this invention to provide a portable wound cleansing device that provides visual indication of how much wound cleansing liquid remains in the device.

It is yet another object of this invention to provide a portable wound cleansing device which provides visual indication that the bottle of fluid has already been used on a prior patient.

It is yet another object of this invention to provide a portable wound cleansing device that prevents spillage of irrigation fluid during procedural interruptions as well as after the procedure.

Accordingly, this invention provides: a portable, manually-operated, wound cleansing liquid dispenser apparatus consisting of: a manually-squeezable saline irrigation solution container for holding and selectively delivering sterile wound irrigation cleansing liquid, the container having a threaded neck; a cap threadably engaging at the threaded neck. The cap carries a nozzle from which wound cleansing liquid issues as a jet in response to manual squeezing of the container. The cap defines a liquid flow path leading to the nozzle; and a splash shield surrounds the nozzle for protecting a user of the apparatus from splashing liquid. Further, a valve is disposed in the flow path. This valve may be configured for opening and closing the flow path in response to manual movement of the splash shield between a first and a second position.

These and other objects of the invention will become apparent to those working in the art by reference to the following description, including the accompanying drawings which illustrate two preferred exemplary embodiments of the invention.

### BRIEF DESCRIPTION OF THE DRAWING FIGURES

FIG. 1 is a diagrammatic side elevation view, partially in cross section, of a wound irrigation apparatus embodying the present invention, and which is shown during its use to clean a wound;

FIG. 2 is an enlarged fragmentary view of a portion of FIG. 1 and showing the apparatus according to this invention;

FIG. 3 is an exploded perspective view of the apparatus seen in FIGS. 1-2A;

FIG. 4 provides a fragmentary cross sectional view of an apparatus as seen in FIGS. 1-3, but with the apparatus shown in a storage or shipping configuration prior to use.

FIG. 5 is an enlarged fragmentary view similar to FIG. 2, but showing an apparatus according to an alternative embodiment of the present invention;

FIG. 6 provides an exploded perspective view of the apparatus seen in FIG. 5;

FIG. 7 is a fragmentary cross sectional view of the apparatus seen in FIGS. 5 and 6, and is illustrated in a use configuration of the apparatus;

FIG. 7A is a fragmentary cross sectional view of the apparatus seen in FIGS. 5 and 6, and is illustrated in an alternative use configuration of the apparatus;

FIG. 8 is a fragmentary cross sectional view of the apparatus seen in FIGS. 5-7, but is shown in a storage or shipping configuration prior to its use; and

FIG. 9 provides a fragmentary cross sectional view of the apparatus according to FIGS. 5-7, in a configuration it may have in the event of an attempt to make an improper use of the apparatus.

### DETAILED DESCRIPTION OF PREFERRED EMBODIMENTS

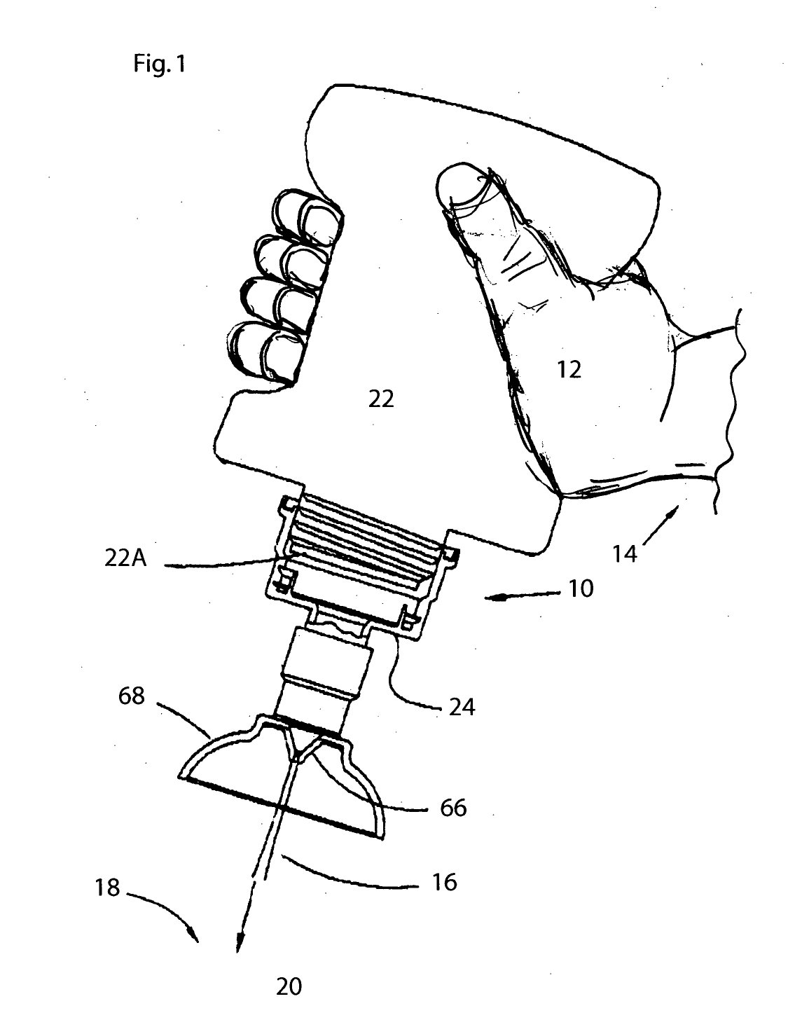

FIG. 1 shows a portable wound cleansing apparatus 10 for dispensing a pressurized (i.e., of selected or controlled velocity) jet or stream of sterile wound cleansing liquid upon and into an open wound. Particularly, the apparatus 10 is generally illustrated in FIG. 1 as it would appear in a use configuration while being held in the squeezing hand 12 of a user 14 (only the hand and wrist of the user 14 being seen in the drawing Figures), who is directing a stream 16 of wound cleaning liquid upon and into a wound 18 of a patient 20 (only a small portion of whom is seen in the drawing Figures).

As is seen in FIGS. 1-4 the apparatus 10 includes a flexible container or plastic bottle 22, which preferable is a standard transparent or translucent plastic saline bottle containing sterile saline irrigation solution, and which is well known and common in the medical field at this time. Threadably attached to the externally threaded neck 22a of the bottle 22 is a dispenser apparatus (generally referenced with the numeral 24) according to a preferred embodiment of this invention.

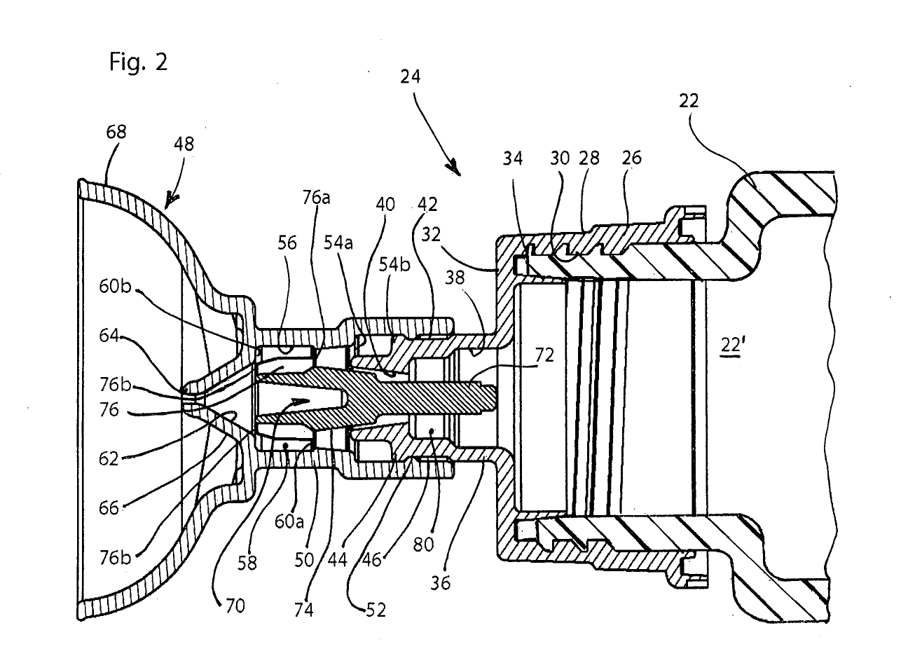

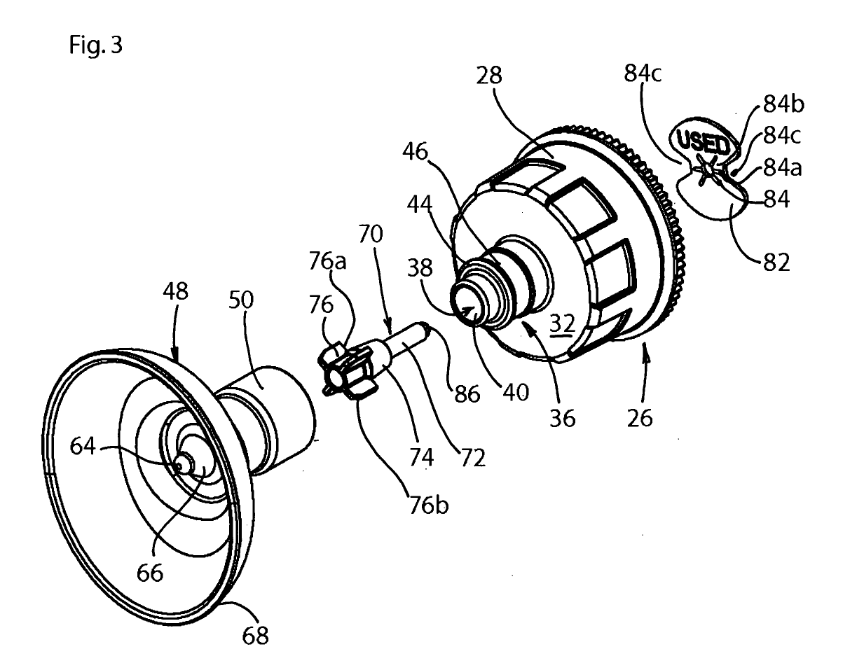

Viewing FIGS. 1-4 in conjunction with one another, and particularly viewing FIGS. 2 and 3, it is seen that dispenser apparatus 24 includes a cap portion 26 defining a collar portion 28 with internally formed (i.e., female) threads 30 for threadably engaging onto the threads of the neck 22A of the bottle 22. Most preferably, the thread 30 is a buttress thread form having a minor diameter of substantially 1.347 inches, and a pitch dimension of 0.164 inch. The female buttress thread shape is preferably formed with the one thread form surface which is disposed toward the container 22 being truly radial (i.e., at an angle of 90° to the axis of the cap) and the other thread form surface at an angle of 46.1° relative to the first surface. Thus, the female thread 30 can threadably engage onto an industry-standard 45° male buttress thread form on the container 22.

Further, the cap portion 26 includes an end wall section 32 inwardly defining an axially extending and slightly tapering sealing collar portion 34. This collar portion 34 is sealingly received inwardly of the neck 22A of the bottle 22, and cooperates with this bottle neck to contain pressurized liquid within the cap 26. That is, the inside of the cap 26 communicates with a cavity 22′ of the bottle 22, and with sterile cleansing liquid in this bottle 22, which is pressurized as seen in FIG. 1 because of the user 14 forcefully squeezing with the hand 12.

Outwardly, the wall section 32 of cap 26 includes a tubular neck section 36 defining a through bore 38. A conical bore section 40 is formed on the through bore 38, and the purpose of this conical section 40 will be explained below. Outwardly, the tubular neck section 36 defines an external cylindrical surface 42, defining a retention collar feature 44 at its distal (i.e., forward) end or termination. Behind the retention collar feature 44, this external surface 42 of the neck section 36 also defines an annular axially extending radial recess 46.

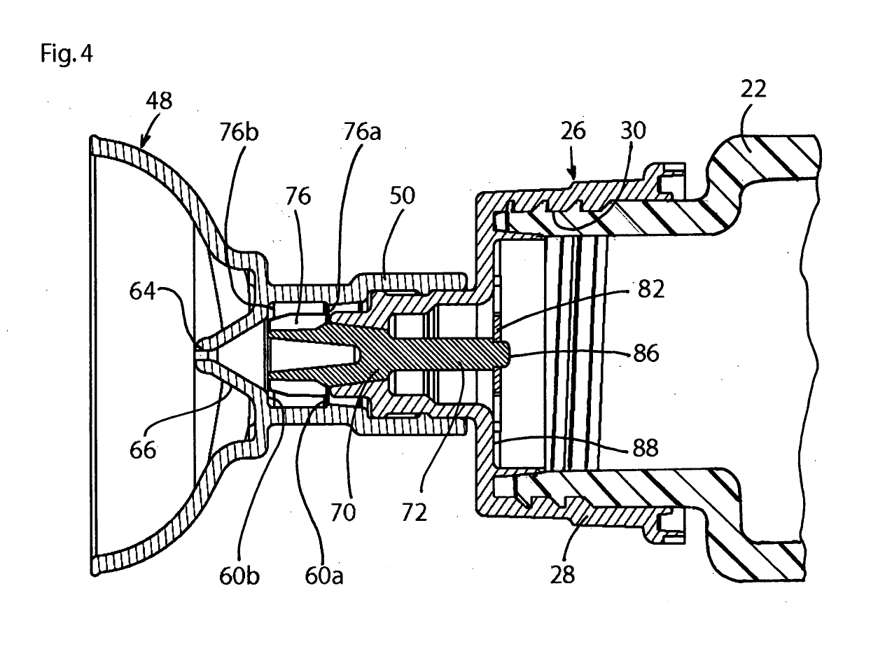

Slidably and captively received on the tubular neck section 36 is a closure and splash guard member, generally indicated with the numeral 48 (hereinafter, “splash guard”). This splash guard 48 includes a tubular section 50 which is slidably and captively received over the neck section 36. Consequently, the splash guard 48 is movable manually between the use position seen in FIGS. 1-3, and a closed (i.e., storage or transport) position seen in FIG. 4. Further considering the splash guard 48 and its tubular section 50, it is seen (particularly in FIGS. 2 and 4) that this tubular section 50 terminates in an annular radially inwardly extending retention collar 52. The retention collar 52 is received on the neck section 36 behind the collar 44 and in the recess 46. In the recess 46 the collar 52 defines a movable sealing interference fit with the tubular section. Accordingly, the splash guard 48 is slidably movable manually between the two positions seen in FIGS. 2 and 4, but is not removable from the neck section 36.

Also seen best in FIGS. 2 and 4, it will be noted that the splash guard 48 tubular section 50 defines a bore 56 (i.e., on which the retention collar 52 appears), and that this bore 56 cooperates with the neck section 36 of the cap 26 to define a chamber 58. The chamber 58 communicates with the chamber 22′ of the bottle 22 via the tubular neck section 36. At the chamber 58, the tubular section 50 defines a pair of oppositely axially disposed annular shoulders (or abutments) 60a and 60b. It will be noted that chamber 58 also communicates via the annular abutment surface 60b to a conical converging section 62 of the bore 56 with a nozzle opening or aperture 64. The nozzle opening 64 is defined by the splash guard 48 on a nozzle protrusion 66 defined within a surrounding annular transparent splash shield portion 68 of the splash guard 48. The splash shield 68 extends radially outwardly of and forwardly of the nozzle opening 64 and nozzle protrusion 66. As is seen in FIG. 1, this splash shield 68 is effective to prevent or reduce liquid from the jet 16 and wound 18 from splashing back toward and upon the user 14.

Turning again to FIGS. 3 and 4, it is seen that movably received in the chamber 58 (that is, in bores 38 and 56 of the neck section 36 and of tubular section 50, respectively) is a valve member 70. This valve member includes a stem portion 72 extending through the bore 38 to (in the position seen in FIG. 4) extend slightly into the chamber 22′ of bottle 22. In the conical bore portion 40, the stem portion 72 defines a matchingly shaped conical valve portion 74. Forwardly of the conical valve portion 74, the valve member 70 includes a plurality of radially outwardly and axially extending fin members 76 (best seen in FIG. 3, and only two of which are seen in FIGS. 2 and 4). These fin members 76 each engage at respective oppositely axially disposed end edges 76a and 76b with the annular abutment surface 60a and 60b. Consequently, the valve member 70 moves axially with the splash guard member 48, and in the position of the splash guard member 48 seen in FIG. 2 a liquid flow path (indicated by the arrowed numeral 80) is maintained from chamber 22′ along bore 38 to chamber 58, along bore 56, past the annular abutment surface 60b (i.e., between the plural fins 76), along the conical section 62, and to nozzle opening 64. Thus, as is seen in FIGS. 1, and 2, liquid can flow from the bottle 22 (i.e., because of the squeezing pressure manually applied by the user, as is seen in FIG. 1) to be ejected from the nozzle opening 64 as a jet or stream of cleansing liquid.

Accordingly, the user of the apparatus 10 may cleanse and irrigate a wound as is seen in FIG. 1, by applying manual pressure to the bottle 22, ejecting a stream or jet of cleansing liquid 16 from the nozzle 64. The transparent splash shield 68 allows the user to see the direction and effect of the jet 16 of cleansing liquid, while substantially reducing back splash of contaminated liquid toward the user of the apparatus 10.

Turning now to FIG. 4, the configuration of the apparatus 10 during transport or storage (i.e., prior to use) is illustrated. In this configuration the splash guard member 48 is disposed axially along the tubular neck 36 toward the cap 26, and is retained in its closed position by the sliding interference fit of the collar portion 52 in recess 46, as illustrated. Further, the valve member 70 is retained in its closed position by the splash guard 48 in a closed position along neck 36, because the annular abutment surface 60a engages against the ends 76a of the fins 76 and holds the valve 70 in its closed position, viewing FIG. 4. Additionally, in this storage or transport configuration of the apparatus 10, an indicator disk member 82 (i.e., a tell-tale member) is received in the chamber 22′ of the bottle 22 and is captively but releasably retained on the stem portion 72. This indicator disk 82 defines a central hole or aperture 84 surrounded by a radially extending plurality of slots 84a. Consequently, between the slots 84a, the disk 82 defines a radial plurality of resilient finger portions 84b. The finger portions 84b captively but releasably engage about an end portion feature 86 (to be further described below) of the stem 72. The disk member 82 also engages against a surrounding shoulder 88 within the cap 24. Further, the disk 84 defines a pair of flow path notches 84c. These flow notches are important because they insure that the disk member 82 does not block liquid flow from the bottle 22 in the event that the apparatus 10 is quickly brought into use (i.e., the splash guard 48 is pulled to its open position essentially simultaneously with the application of squeezing pressure on the bottle 22).

When the splash guard member 48 is moved from its first or closed position of FIG. 4, and to its second or opened position as seen in FIG. 2, then a snap fit feature 86 of the stem portion 72 which is captively received in this aperture 84 among the finger portions 84b is pulled from the disk 82. Because the disk member 82 is engaged against the shoulder 88, it is then released into the chamber 22′, thereafter clearly indicating that the apparatus 10 has been used.

Once the apparatus 10 has been thus opened and used or prepared for use, the tell-tale aspect of the member 82 comes into play. Because the member 82 is dislodged from the end of stem 72, and because this member is preferably formed of plastic having a specific gravity slightly less than water (i.e., less than saline solution), the member 82 floats freely on the surface of any cleansing liquid remaining in the apparatus 10. Thus, the member 82 is preferably made of a plastic which is brightly colored and easily visible. And, the presence of the floating member 82 on the liquid in an apparatus 10 indicates that the apparatus has been used or opened previously, and is to be used only for the patient for which it was opened, and is not to be used on a subsequent different patient. Again, and in view of the above, it will be understood that a user of the apparatus 10 preparatorily grasps the splash shield 48 and pulls it outwardly along the neck 36 from its position of FIG. 4 and to the position of FIG. 2. This preparatory move of the splash shield 48 allows squeezing pressure to move liquid past valve member 70 and along flow path 80 so that the user can discharge a stream of cleansing liquid upon and into a wound as is seen in FIG. 1. And this preparatory move of the splash shield member 48 also and simultaneously releases the disk member 82 inside of the bottle 22 so that thereafter the apparatus is recognizable as “used,” and not suitable for use on another patient.

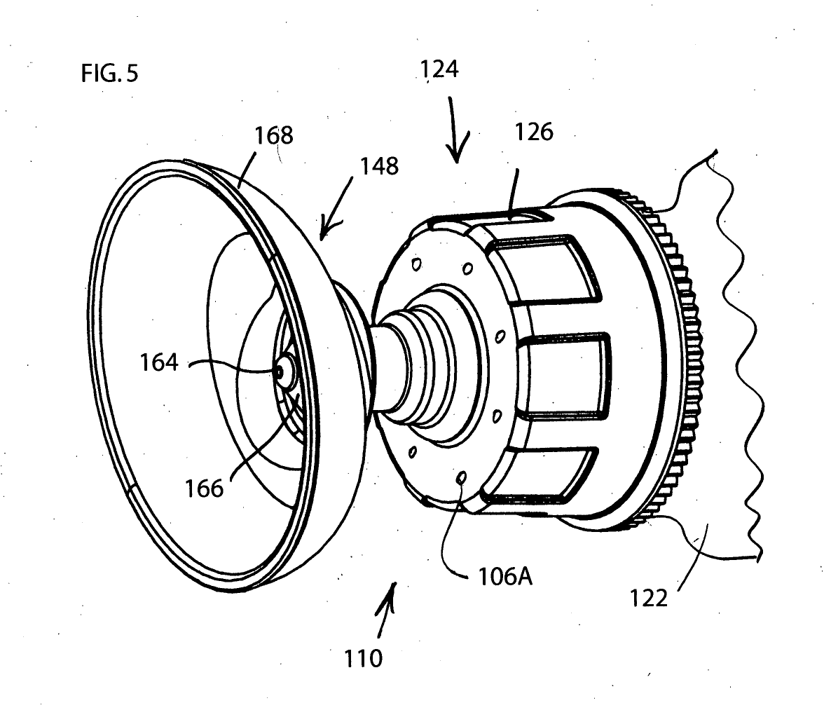

Turning now to FIGS. 5-8, an alternative embodiment of the present invention is illustrates. In order to obtain reference numerals for use in describing the alternative embodiment of FIGS. 5-8 features of this embodiment which are the same as or analogous to those illustrated and described earlier with respect to FIGS. 1-4 are referenced on FIGS. 5-8 using the same numeral increased by one-hundred (100).

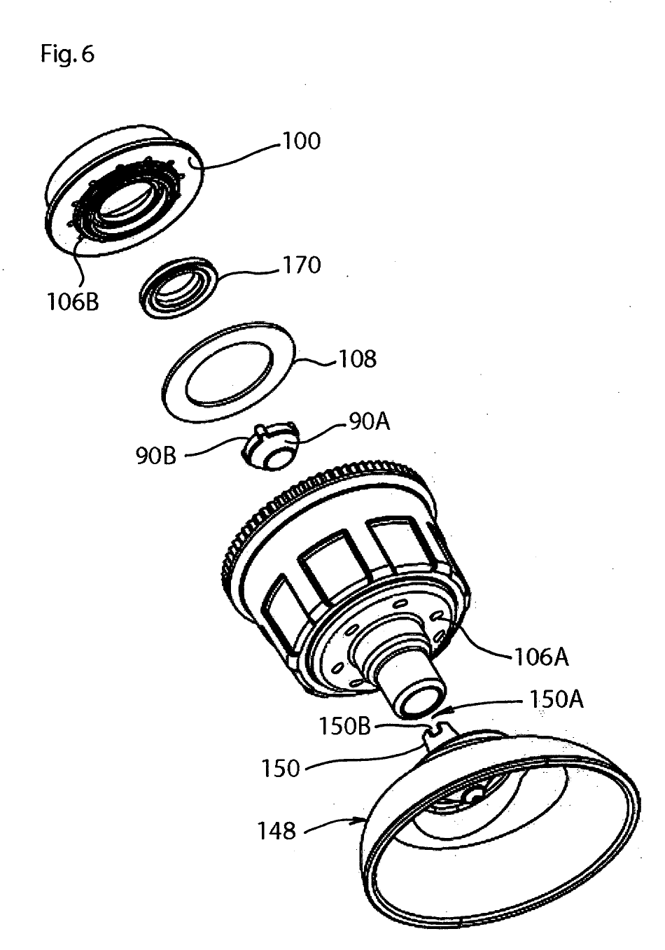

FIG. 5 is similar to FIG. 2, and shows the apparatus 110 during its use to discharge a jet or stream of cleansing liquid 116. FIG. 6 illustrates the apparatus 110 in cross section, and shows that this apparatus includes a flexible container or plastic bottle 122. Threadably attached to the externally threaded neck 122a of the bottle 122 is a dispenser apparatus 124 according to an alternative embodiment of this invention. The dispenser apparatus 124 includes a cap portion 126 defining a collar portion 128 with internally formed (i.e., female) threads 130 for threadably engaging onto the threads of the neck 122A of the bottle 122. In this alternative embodiment, the cap portion 126 includes an end wall section 132, including a tubular neck section 136 defining a through bore 138. The bore portion 138 includes an inwardly tapering portion 138a, leading to a slightly enlarged generally cylindrical section 138b. An annular disk member 100 is received against the end wall 132, and defines a slightly tapering sealing collar portion 134, which is sealingly received inwardly of the neck 122A of the bottle 122. That is, this collar portion 134 sealingly cooperates with the bottle neck 122A of the bottle 122.

The disk member 100 cooperates with the wall 132 of the cap member 126 to define a pair of radially spaced radially extending and annular recesses 102A and 102B. Communicating with the inner one (i.e., 102A) of these annular recesses, the disk member 100 cooperates with the cap member 126 to define also a chamber 104 having a conical front wall section 138c (i.e., defined by a conical portion of the bore 138). In the annular recess 102A, and captively received between the cap member 126 and the disk member 100 is a non-reversion resilient slit-valve member 170. In contrast to the poppet valve type of construction used for the non-reversion valve 70, the slit-valve member 170 employs a resilient disk 170A defining at least one slit 170B.

Movable received captively in the chamber 104 is a flow-responsive safety valve 90 having a poppet type of valve member 90A confronting and sealingly engageable with the conical front wall section 138C. Opposite to the front wall section 138C, the valve member 90a defines a plurality of axially extending legs 90B. These legs 90B are engageable with an outer peripheral portion of the non-reversion valve member 170 and define flow path sections therebetween so that the safety valve member 90A does not sealingly engage with the non-reversion valve member.

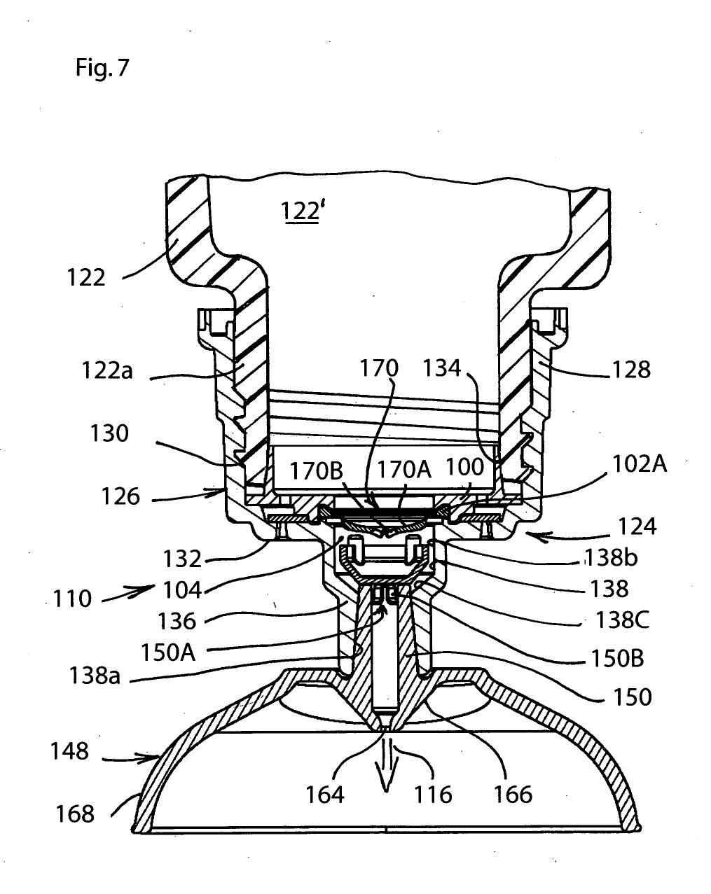

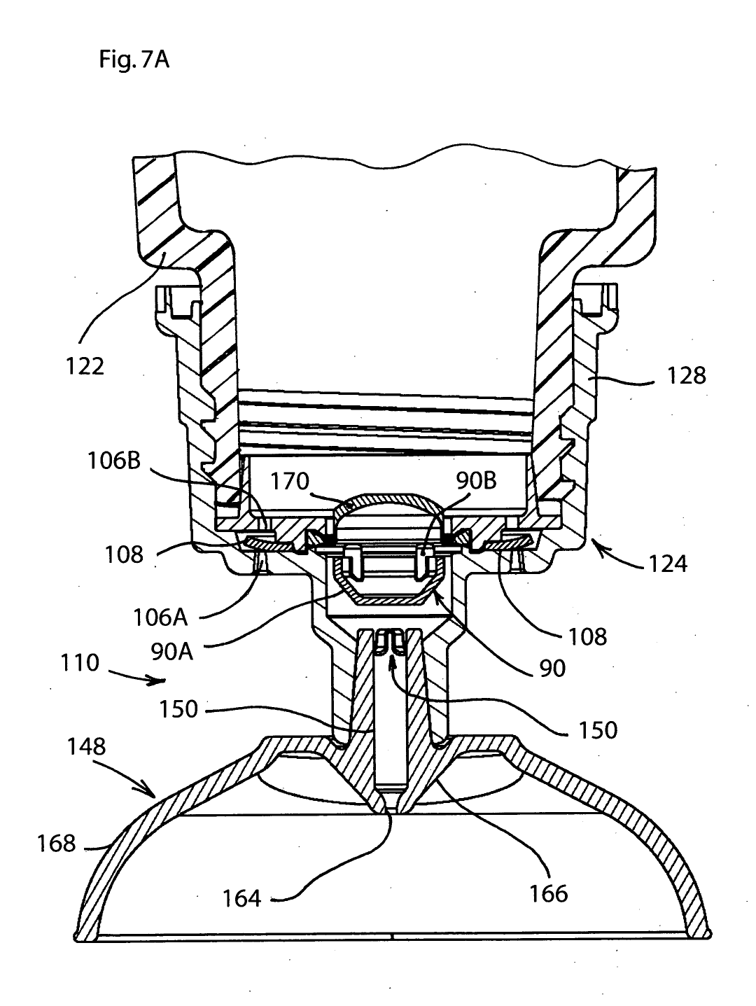

As is further seen particularly in FIG. 7, a splash guard member 148 is sealingly retained in the forward inwardly tapered portion 138A of bore 138 by the sealing and retaining cooperation of an outwardly tapered stem portion 150 of the splash guard member 148. At its inward distal end termination, this tapered stem portion 150 defines a crenellated end structure 150A, defining plural flow path crenellations 150B. As is seen in FIG. 7, when the splash guard member 148 is properly and fully inserted and sealingly seated at its stem portion 150 in the bore portion 138A, then the crenellation feature 150A engages against the safety valve member 90A, keeping this valve member from engaging against the conical wall section 138C. Accordingly, a flow path 180 is maintained through valve member 170 (which is pressure and flow responsive to open when the bottle 122 is squeezed with sufficient force), between the legs 90B of the valve member 90A, along chamber 104, through the crenellations 150B, and to the nozzle orifice 164. Thus, as is seen in FIG. 7, a stream of cleansing liquid issues from the orifice 164 on nozzle protrusion 166 within the transparent splash shield portion 168.

Now, when the user of the apparatus 110 relaxes manual squeezing on the bottle 122, the resilient nature of the bottle itself results in a slight negative pressure being developed within the bottle 122. However, this slight negative pressure is not sufficient to open the non-reversion slit-type valve 170. Thus, back flow of contaminated liquid from the front surface of the splash shield 168 is substantially avoided. However, in order to provide for the bottle 122 to aspirate ambient air (which assists in further discharging cleansing liquid from the bottle 122 upon a subsequent squeezing of the bottle) the cap member 126 and disk member 100 each define respective ones of a plurality of vent passages 106A and 106B. And, within the annular recess 104B a resilient annular disk valve member 108 is disposed to cover the passages 106A. Thus, when the bottle 122 contains a negative (i.e., sub-ambient) pressure, as is seen in FIG. 7A, the disk valve 108 is lifted slightly off the passages 106A, and allows the bottle to aspirate ambient air. On the other hand, as is seen in FIG. 7, when the bottle 122 is being squeezed the disk valve 108 covers the passages 106A, preventing cleansing liquid from being squeezed out of the vent passages. Thus, it is seen that the disk valve 108 serves as a check valve.

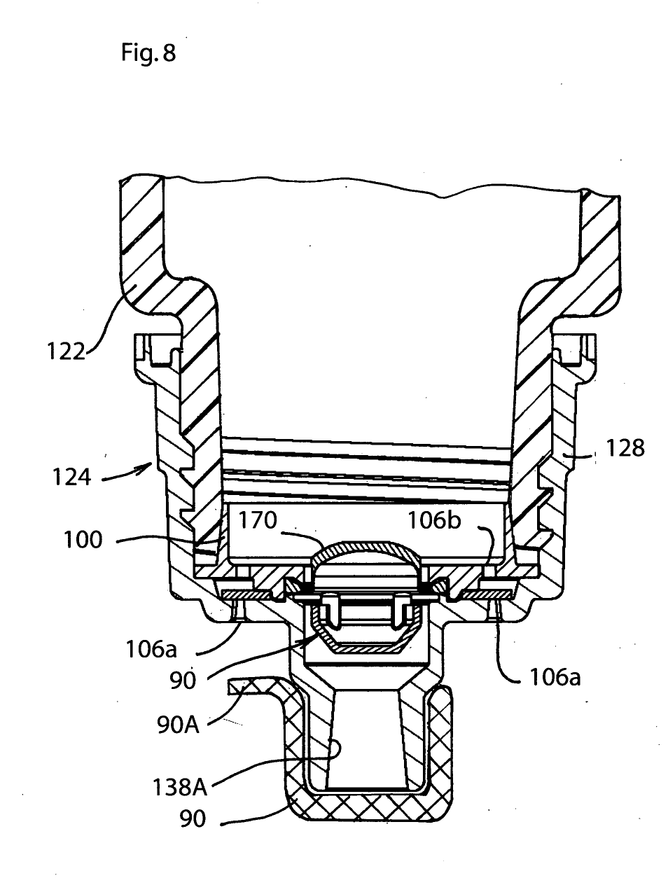

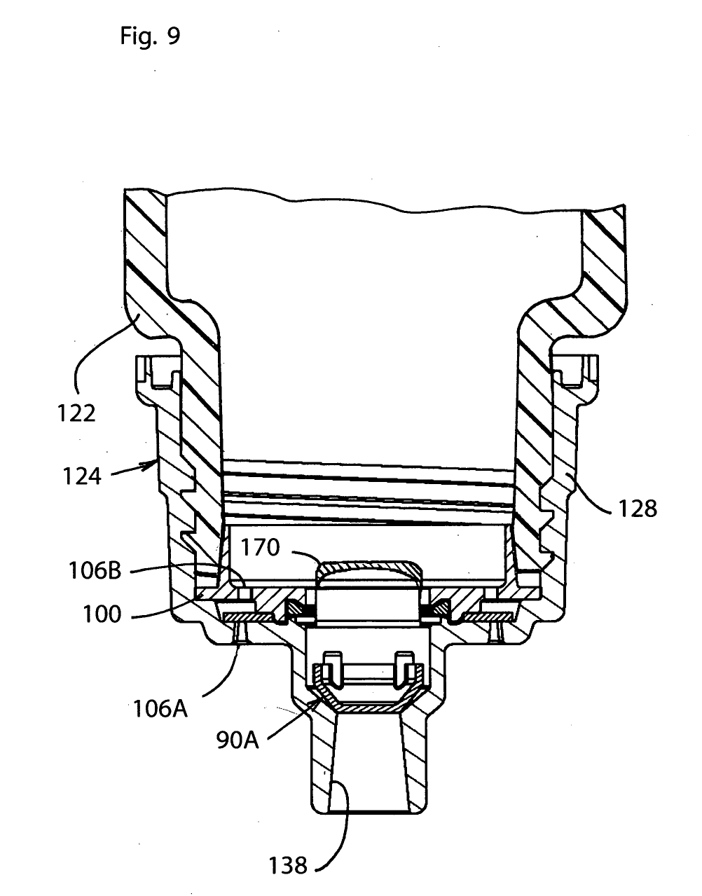

Viewing now FIGS. 8 and 9, and considering first FIG. 8, this Figure shows the apparatus 110 during a storage or shipping condition. That is, during storage and shipping, a closure member 90 covers the tubular portion 136, and the opening of the bore 138. In order to use the apparatus 110, the user grasps an extending tang 90A of the cap 90 and pulls to fracture the cap, thus ripping this cap off. The user then inserts the splash shield 148 as explained earlier in order to place the apparatus 110 in the use configuration. However, in the event that a user attempts to use the apparatus improperly and without the splash shield 148, as is illustrated hypothetically in FIG. 9, then the safety valve 90 prevents such misuse. That is, the safety valve 90 is pressure and flow responsive, and engages sealingly upon the tapered wall section 138C to prevent cleansing liquid outflow from the bottle 122, as is seen in FIG. 9. Accordingly, the apparatus 110 cannot be used by the user until the splash shield 148 is properly installed.

The present invention is not limited to the embodiments described above, and it is to be understood that the invention is limited only by the spirit and scope of the appended claims, which provide a definition of the invention.

1. A portable, manually-operated, wound cleansing liquid dispenser apparatus consisting of:
a manually-squeezable irrigation solution container for holding and selectively delivering sterile wound irrigation cleansing liquid, said container having a threaded neck;
a cap threadably engaging at said threaded neck, said cap carrying a nozzle from which said wound cleansing liquid issues as a jet in response to manual squeezing of said container, said cap defining a liquid flow path leading to said nozzle;
a splash shield surrounding said nozzle for protecting a user of said apparatus from splashing liquid; and
a valve disposed in said flow path for opening and closing said flow path.

2. The dispenser apparatus of claim 1 wherein said valve opens and closes said flow path in response to manual movement of said splash shield between a first and a second position.

3. The dispenser apparatus of claim 1 wherein said threaded neck of said container defines an external thread, and said cap including a collar portion internally defining a matching thread.

4. The dispenser apparatus of claim 3 wherein said matching thread of said cap has a minor diameter of substantially 1.347 inches, and a pitch dimension of substantially 0.164 inch.

5. The dispenser apparatus of claim 4 wherein said matching thread is a buttress configuration thread and has a first 90° radial surface and a second surface disposed at about 45° to said first surface.

6. The dispenser apparatus of claim 1 wherein said valve includes a valve member moving between respective first and second locations in response to manual movement of said splash shield between said first and second positions.

7. The dispenser of claim 6 wherein said cap defines a bore forming a part of said flow path and including a tapering bore section, said valve member including a tapering portion which in one of said first and second locations of said valve member sealingly engages on said tapering bore section.

8. The dispenser apparatus of claim 7 wherein said cap bore defines a shoulder disposed toward said container, said valve member including a stem portion having an end termination feature substantially aligned with said shoulder in said first position of said splash shield and in said respective first location of said valve member, an indicator member releasably carried on said stem portion end termination feature and engaging said shoulder, whereby when said valve member is moved to said second location in response to manual movement of said splash shield to said second position, said indicator is dislodged from said end termination feature and is freed inside of said container.

9. The dispenser apparatus of claim 8 wherein said indicator is disk-like and brightly colored to be visible when freed within said container, whereby opening of said dispenser apparatus is irreversibly indicated by freeing of said indicator within said container.

10. The dispenser apparatus of claim 8 wherein said indicator is disk-like and includes a sufficient number of peripherally disposed circumferentially spaced apart projections for preventing said indicator from being retained by surface tension.

11. The dispenser apparatus of claim 8 wherein said disk-like indicator defines at least one flow notch, whereby said indicator is prevented by said flow notch from ever acting as a valve member preventing flow of liquid from said container via said cap.

12. The dispenser apparatus of claim 7 wherein said valve member further includes a plurality of axially extending legs for forming therebetween a part of said flow path in one of said first and second locations for said valve member.

13. The dispenser apparatus of claim 1 wherein said valve includes a non-reversion valve member which substantially prevents flow of liquid along said flow path from said nozzle toward said container.

14. The dispenser apparatus of claim 13 wherein said non-reversion valve member includes a resilient portion defining at least one slit, said slit opening in response to manual squeezing of said container and liquid flow therefrom toward said nozzle, said resilient portion bowing toward said container and closing said slit to substantially prevent liquid flow along said flow path from said nozzle toward said container.

15. The dispenser apparatus of claim 13 wherein said cap further includes a passage extending between ambient and said irrigation solution in said container, a pressure responsive aspiration valve disposed in said passage for allowing ambient air to enter said container when a sub-ambient pressure exists therein.

16. The dispenser of claim 15 wherein said cap defines plural said passages annularly arrayed and circumferentially spaced apart, and said valve includes an annular resilient disk valve member.

17. The dispenser apparatus of claim 1 wherein said splash shield member is separable from said cap in said first position.

18. The dispenser apparatus of claim 17 in which said valve member prevents dispensing of liquid from said container via said cap when said splash shield is separated from said cap.

19. The dispenser apparatus of claim 18 wherein said cap defines a bore forming a part of said flow path and including a tapering bore section, wherein said valve includes a manually-operated valve member moving between respective first and second locations in response to manual movement of said splash shield between said first and second positions; said manually-operated valve member including a tapering portion which in one of said first and second locations of said manually-operated valve member sealingly engages on said tapering bore section; and wherein said splash shield is defined by a splash shield member also including a stem portion sealingly receivable into said bore of said cap in said second position of said splash shield, said splash shield member stem including an end feature engaging against said manually-operated valve member in said second position of said splash shield to prevent said manually-operated valve member from engaging against said tapering section of said bore, whereby said apparatus may discharge pressurized liquid from said nozzle in response to manual squeezing of said container only so long as said splash shield member stem portion is sealingly received into said bore of said cap.

20. The dispenser apparatus of claim 6 wherein said cap defines a bore forming a part of said flow path and including a tapering bore section, wherein said valve includes a manually-operated valve member moving between respective first and second locations in response to manual movement of said splash shield between said first and second positions; said manually-operated valve member including a tapering portion which in one of said first and second locations of said manually-operated valve member sealingly engages on said tapering bore section; and wherein said manually-operated valve member further includes a plurality of axially extending legs for forming therebetween a part of said flow path in one of said first and second locations for said manually-operated valve member; wherein said valve also includes a non-reversion valve member which substantially prevents flow of liquid along said flow path from said nozzle toward said container, said plurality of valve member legs of said manually-operated valve member extending toward engagement with said non-reversion valve member; and wherein said manually-operated valve member is also responsive to pressurized liquid resulting from squeezing of said container to sealingly engage against said tapering section of said bore so as to also serve as a safety valve by moving to one of said first and second locations and into sealing engagement with said tapering bore section so as to prevent liquid flow along said flow path, whereby in the event that said splash shield is absent from said dispenser apparatus then liquid flow from said container via said cap is prevented.

21. The dispenser apparatus of claim 20 wherein said splash shield is defined by a splash shield member also including a stem portion sealingly receivable into said bore of said cap in a second position of said splash shield, said splash shield member stem including an end feature engaging against said manually-operated valve member to prevent said manually-operated valve member from engaging against said tapering section of said bore, whereby said apparatus may discharge pressurized liquid from said nozzle in response to manual squeezing of said container only so long as said splash shield member stem portion is sealingly received into said cap bore.

22. The dispenser apparatus of claim 20 wherein said cap further includes a passage extending between ambient and said irrigation solution in said container, a pressure responsive aspiration valve disposed in said passage for allowing ambient air to enter said container when a sub-ambient pressure exists therein.

23. The dispenser of claim 22 wherein said cap defines plural said passages annularly arrayed and circumferentially spaced apart, and said valve includes an annular resilient disk valve member.

24. A dispenser cap for combination with a standard irrigation solution bottle to make a portable wound irrigation dispenser for discharging a cleansing liquid jet when the bottle is inverted and manually squeezed, said cap comprising:
a collar portion defining a female thread matching the thread on said standard irrigation solution bottle;
a wall portion spanning an end of said cap opposite to said bottle;
a tubular neck section extending from said wall and defining a through bore defining a flow path communicating irrigation solution from said bottle;
a splash shield member carried by said tubular neck section and including a nozzle for directing said jet of cleansing liquid; and
a valve for opening and closing said flow path.

25. The dispenser cap of claim 24 wherein said splash shield member is manually movable axially between a first and a second position, and said valve opens and closes said flow path in response to movement of said splash shield member between said first and said second positions.

26. The dispenser cap of claim 24 wherein the container includes a threaded neck defining an external thread, and said cap collar portion internally defines a matching thread; wherein said matching thread has a minor diameter of substantially 1.347 inches, and a pitch dimension of substantially 0.164 inch.

27. The dispenser cap of claim 24 wherein said valve includes a valve member moving between respective first and second locations in response to manual movement of said splash shield between said first and second positions; and said cap includes an axially extending tubular neck portion defining a through bore forming a part of said flow path and including a tapering bore section, said valve member including a tapering portion which in one of said first and second locations of said valve member sealingly engages on said tapering bore section.

28. The dispenser cap of claim 27 wherein said cap bore defines a shoulder disposed toward an open end of said cap, said valve member including a stem portion having an end termination feature substantially aligned radially with said shoulder in said first position of said splash shield member and in said respective first location of said valve member, an indicator member releasably carried on said stem portion end termination feature and in a first location of said valve member engaging said shoulder, whereby when said valve member is moved to said second location in response to manual movement of said splash shield to said second position, said indicator is dislodged from said end termination feature and is freed inside of said container, thus irreversibly indicating that the apparatus has been used.

29. The dispenser cap of claim 28 wherein said indicator is disk-like and defines at least one flow notch, whereby said disk-like indicator is prevented by said flow notch from ever acting as a valve member preventing flow of liquid from said container via said cap.

30. The dispenser apparatus of claim 28 wherein said indicator includes a sufficient number of peripherally disposed circumferentially spaced apart projections for preventing said indicator from being retained by surface tension.

31. The dispenser cap of claim 24 wherein said valve member further includes a plurality of axially extending legs for forming therebetween a part of said flow path in one of said first and second locations for said valve member; and said cap further includes a non-reversion valve member disposed across said bore and substantially preventing flow of liquid along said flow path from said nozzle toward said bottle.

32. The dispenser cap claim 31 wherein said non-reversion valve member includes a resilient wall portion spanning said bore and defining at least one slit, said slit opening in response to manual squeezing of said container and liquid flow therefrom toward said nozzle, said resilient portion bowing toward said container and closing said slit to substantially prevent liquid flow along said flow path from said nozzle toward said container.

33. The dispenser apparatus of claim 32 wherein said cap further includes a passage extending between ambient and said irrigation solution in said container, a pressure responsive aspiration valve disposed in said passage for allowing ambient air to enter said container when a sub-ambient pressure exists therein.

34. The dispenser of claim 33 wherein said cap defines plural said passages annularly arrayed and circumferentially spaced apart, and said valve includes an annular resilient disk valve member.

35. The dispenser cap of claim 24 wherein said splash shield is defined by a splash shield member also including a stem portion sealingly receivable in said second position of said splash shield into said bore of said cap, said stem of said splash shield member including an end feature engaging against said manually-operated valve member in said second position of said splash shield to prevent said manually-operated valve member from engaging against said tapering section of said bore, whereby said apparatus may discharge pressurized liquid from said nozzle in response to manual squeezing of said container only so long as said stem portion of said splash shield member is sealingly received into said bore of said cap.

36. A method of providing a dispenser cap for combination with a standard irrigation solution bottle to make a portable wound irrigation dispenser, the portable wound irrigation dispenser being useful for discharging a cleansing liquid jet into a wound when the bottle is inverted and manually squeezed, said method comprising steps of:
providing a collar portion defining a female thread matching the thread on said standard irrigation solution bottle;
spanning one end of said collar portion with a wall portion to said bottle;
extending a tubular neck section axially from said wall portion and forming in said neck section a through bore defining a flow path communicating irrigation solution from said bottle;
providing a splash shield member disposed upon said tubular neck section and including a nozzle for directing a jet of cleansing liquid, providing for said splash shield member to be manually movable relative to said collar portion between a first and a second position; and
including in said flow path a valve for opening and closing liquid flow therein in response to movement of said splash shield member between said first and said second positions.

37. The method of claim 36 further including the steps of:
providing for said cap collar portion to internally define a thread;
configuring said thread with a minor diameter of substantially 1.347 inches, and a pitch dimension of substantially 0.164 inch.

38. The dispenser cap of claim 36 further including the steps of;
providing for said dispenser cap to carry in indicator disposed within said bottle; and
in response to movement of said splash shield member between said first and said second positions releasing said indicator from said dispenser cap within said bottle, thus irreversibly indicating that the wound irrigation apparatus has been used.

39. A dispenser cap for combination with a standard irrigation solution bottle to make a portable wound irrigation dispenser for discharging a cleansing liquid jet when the bottle is inverted and manually squeezed, said cap comprising:
a collar portion defining a female thread matching the thread on said standard irrigation solution bottle;
a wall portion spanning an end of said cap opposite to said bottle;
a tubular neck section extending from said wall and defining a through bore defining a flow path communicating irrigation solution from said bottle; and
a splash shield member carried by said tubular neck section and including a nozzle for directing said jet of cleansing liquid.
